(19) United States
(12) Patent Application Publication
(10) Pub. No.: US 2026/0078418 A1
(43) Pub. Date: Mar. 19, 2026

(54) A METHOD OF MAKING AN ETHANOL COMPOSITION AND A SEPARATE GALACTOSE COMPOSITION BY FERMENTATION

(71) Applicant: West Cork Distillers Limited, Skibbereen (IE)

(72) Inventors: Albert O'Sullivan-Greene, Castletownbere (IE); John Harrington, Clonakilty (IE); Valda Margaret Smith, Timoleague (IE)

(21) Appl. No.: 19/130,662

(22) PCT Filed: Nov. 20, 2023
(86) PCT No.: PCT/EP2023/082448

§ 371 Date: May 16, 2025

(30) Foreign Application Priority Data
Nov. 18, 2022 (EP) 22208444.4

(51) Int. Cl. C12P 19/02; B01D 15/08; B01D 21/26; B01D 61/02; B01D 61/04; B01D 69/02; C12N 9/38; C12P 7/06

(52) U.S. Cl. CPC C12P 19/02; B01D 15/08; B01D 21/262; B01D 61/025; B01D 61/04; B01D 69/02; C12N 9/2471; C12P 7/06; C12Y 302/01023; B01D 2325/02831

(57) ABSTRACT

A method of making an ethanol composition and a separate galactose composition by fermentation is described. The method does not require wheat substrates and does not require a distillation step to separate out the ethanol. The methods comprises treating the lactose containing substrate with a beta-galactosidase enzyme to provide an enzyme treated substrate in which at least some of the lactose in the substrate has been enzymatically converted into glucose and galactose, fermenting in a fermentation medium comprising the enzyme-treated substrate a glucose fermenting yeast to provide a fermentation broth containing ethanol and galactose, and separating the fermentation broth into an ethanol composition and a separate galactose composition. The fermentation broth is generally separated by reverse osmosis into a retentate and a permeate, in which the permeate is the ethanol composition and the retentate is the galactose composition.

### FIELD OF THE INVENTION

The present invention relates to a method of making an ethanol composition and a separate galactose composition by fermentation.

### BACKGROUND TO THE INVENTION

Currently, ethanol is primarily produced from grain carbohydrate sources, e.g. glucose, maltose, maltotriose, by fermentation. Lactose sources are also used in fermentation of bio-ethanol where the ethanol separation occurs via distillation as opposed to filtration. Distillation as a method of separating lactose derived ethanol is energy intensive and the resultant product is classified as a spirit as opposed to a fermentation/beer. Current methods of lactose fermentation do not utilise the Galactose sugars into a commercialised product.

U.S. Pat. No. 8,252,350 describes a method of ethanol recovery from a fermentation broth that comprises removing aliquots of a fermentation broth from a reactor while the fermentation is active. The aliquots that are removed have a low alcohol level (1-3% ABV) and are concentrated by a first reverse osmosis (RO) separation stage to produce a permeate with an ABV of about 6%, and then a second RO separation stage or a pervaporation treatment to provide a permeate containing 12% to 17% ABV. The constant removal of alcohol from the active fermentation broth improves the fermentation process.

U.S. Pat. No. 6,057,135 describes a process for making D-tagatose from cheese whey or milk in which lactose in the milk/whey is hydrolysed with lactase to produce glucose and galactose, the treated milk/whey is fermented to convert the glucose into ethanol, the galactose is separated from the ethanol by pervaporation of the ethanol, and the galactose is treated with L-arabinose isomerase to produce D-tagatose. The ethanol is further concentrated by distillation. Pervaporation is an energy intensive process and requires constant cleaning and replacement of the pervaporation membranes. In addition, pervaporation involves a phase change in the ethanol (similar to distillation) which often prevents the resultant alcohol being classed as a fermentate.

Galactose is a simple form of sugar. It is found in nature as one of the three monosaccharides, the other two being Fructose and glucose. It is widely used in the pharma, food and supplement industries. Methods for the industrial production of galactose are limited, and many employ wheat based substrates and/or expensive chemical processes.

It is an object of the invention to provide a process for producing ethanol that does require use of grain carbohydrate sources and does not require energy intensive distillation or pervaporation processes.

### SUMMARY OF THE INVENTION

The Application has addressed the problems of the prior art by providing a process for producing ethanol and galactose by fermentation that does not require grain carbohydrate substrates and does not require energy intensive phase-change processes such as pervaporation or distillation. The method employs a lactose substrate instead of a grain substrate, for example a dairy whey stream that is rich in lactose. The lactose substrate is treated enzymatically to convert the lactose into glucose and galactose, and then fermented with a suitable yeast to provide a fermentation broth containing ethanol and galactose. The fermentation broth is then separated by a suitable separation technology such as reverse osmosis to remove the ethanol from the fermentation broth leaving behind a galactose rich composition, which may then be further treated by, e.g. demineralisation to enrich for galactose. The process results in an ethanol stream containing up to 20% or even 25% ethanol by volume that does not require processing comprising a phase change (e.g. does not require distillation or pervaporation) and therefore can be classed as a fermentate, and a galactose composition which contains high levels of galactose that can be produced from a non-plant source.

In a first aspect, the invention provides a method of making an ethanol composition and a separate galactose composition by fermentation and comprising the steps of:
proving a lactose containing substrate;
treating the lactose containing substrate with an enzyme (e.g. a beta-galactosidase enzyme) to provide an enzyme treated substrate in which at least some of the lactose in the substrate has been enzymatically converted into glucose and galactose;
fermenting in a fermentation medium comprising the enzyme-treated substrate a glucose fermenting yeast to provide a fermentation broth containing ethanol and galactose; and
separating the fermentation broth into an ethanol composition and a separate galactose composition.

In any embodiment, the fermentation broth is separated by reverse osmosis into a retentate and a permeate, in which the permeate is the ethanol composition and the retentate is the galactose composition.

In any embodiment, the fermentation broth is separated into a retentate and a permeate by a single reverse osmosis separation stage.

In any embodiment, the fermentation step comprises converting at least 60%, 70%, 80%, 90%, 95% or 99% of the glucose into ethanol (v/v).

In any embodiment, the galactose composition is treated to enrich the galactose content of the galactose composition.

In any embodiment, the galactose composition is treated to provide a galactose composition containing at least 9% galactose by volume.

In any embodiment, the galactose composition is treated to provide a galactose composition containing at least 60%, 70%, 80% or 90%, 92%, 95% or 98% galactose by volume.

In any embodiment, the galactose composition is treated by demineralisation.

In any embodiment, the galactose composition is treated by one or more of nano-filtration, diafiltration, electrical dialysis, ion exchange chromatography, and size exclusion chromatography. In any embodiment, the galactose composition is treated with a filtration step and a chromatography step.

In any embodiment, the ethanol composition is treated to de-water the ethanol composition.

In any embodiment, the lactose substrate comprises at least 3%, 4%, 5%, 10%, 12%, 15%, 17% or 20% lactose by volume.

In any embodiment, the lactose substrate comprises less than 3%, 2%, 1% or 0.5% glucose by volume.

In any embodiment, the lactose substrate comprises less than 3%, 2%, 1% or 0.5% galactose by volume.

In any embodiment, the lactose substrate is substantially free of glucose and/or galactose.

In any embodiment, the lactose substrate is dairy whey or a dairy whey derivative.

In any embodiment, the lactose substrate is a whey derivative that has been treated to remove protein.

In any embodiment, the glucose fermenting yeast is a Saccharomyces spp.

In any embodiment, the glucose fermenting yeast is a strain of Saccharomyces cerviscae.

In any embodiment, the method comprises a step of removal of yeast cells from the fermentation broth prior to the step of separation of the treated fermentation broth.

In any embodiment, the step of removal of yeast cells from the fermentation broth comprises centrifugation of the fermentation broth (e.g. where the supernatant is then subjected to RO treatment). The applicant has surprisingly discovered that pre-treatment of a fermentation broth to remove yeast cells allows the treated fermentation broth to be treated by reverse osmosis to produce a permeate having high a % ABV, for example 10 to 25%, without the need for energy intensive phase change treatments such as distillation or pervaporation.

In any embodiment, the treated fermentation broth (e.g. supernatant) has an ABV of 1% to 15%.

In any embodiment, the treated fermentation broth (e.g. supernatant) has an ABV of 1% to 10%.

In any embodiment, the treated fermentation broth (e.g. supernatant) has an ABV of 5% to 10%.

In any embodiment, the treated fermentation broth (e.g. supernatant) has an ABV of greater than 3%.

In any embodiment, the enzyme treated substrate has a pH of about 6-7.

In any embodiment, the method does not comprise a distillation step.

In any embodiment, the method does not comprise a pervaporation step.

In any embodiment, the production of the ethanol composition does not comprise a distillation or pervaporation step.

In any embodiment, the ethanol composition produced according to the method of the invention has an ABV of 5% to 25%.

In any embodiment, the ethanol composition produced according to the method of the invention has an ABV of 5% to 20%.

In any embodiment, the ethanol composition produced according to the method of the invention has an ABV of 10% to 25%.

In any embodiment, the ethanol composition produced according to the method of the invention has an ABV of 15% to 25%.

In any embodiment, the ethanol composition produced according to the method of the invention has an ABV of 10% to 20%.

In any embodiment, the ethanol composition produced according to the method of the invention has an ABV of 15% to 20%.

In any embodiment, the enzyme treatment comprises converting at least 50%, 60%, 70%, 80%, 90%, 95% or substantially all of the lactose into glucose and galactose.

In another aspect, the invention provides an ethanol composition obtained by a method of the invention.

In another aspect, the invention provides a galactose composition obtained by a method of the invention. In any embodiment, the galactose composition comprises at least 50%, 60%, 70%, 80%, 90%, 95% galactose. In any embodiment, the galactose composition is substantially free of protein, other sugars, and/or minerals.

In another aspect, the invention provides a galactose composition and an ethanol composition obtained by a method of the invention.

Other aspects and preferred embodiments of the invention are defined and described in the other claims set out below.

### BRIEF DESCRIPTION OF THE FIGURE

FIG. 1 is a flow diagram illustrating one embodiment of the method of the invention.

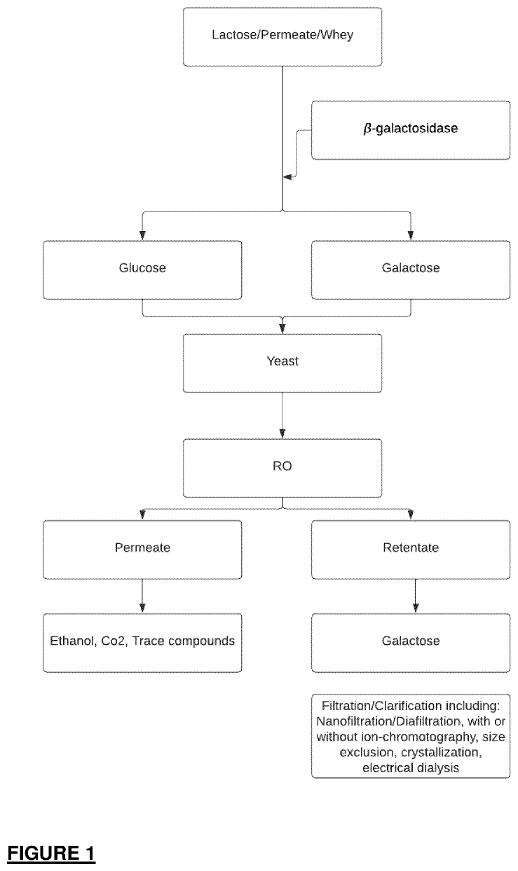

### DETAILED DESCRIPTION OF THE INVENTION

All publications, patents, patent applications and other references mentioned herein are hereby incorporated by reference in their entireties for all purposes as if each individual publication, patent or patent application were specifically and individually indicated to be incorporated by reference and the content thereof recited in full.

### Definitions and General Preferences

Where used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein the term “comprising” is inclusive or open-ended and does not exclude additional, unrecited integers or method/process steps.

As used herein, the term “lactose containing substrate” refers to a composition that contains at least 2% lactose by volume. The substrate also typically contains little or no glucose or galactose. Typically, the lactose substrate is a whey or a whey derivative that is enriched in lactose and/or depleted in protein. The lactose substrate may be demineralised. The whey may be a diary whey, for example sweet whey or acid whey, or may be a whey stream such as a why permeate or a delactosed whey. Whey is a by-product of cheesemaking and is widely available in large quantities in dairy countries such as the US, Ireland, Denmark, and New Zealand.

As used herein, the term “beta-galactosidase enzyme” refers to an exoglycosidase which hydrolyzes the β-glycosidic bond formed between a galactose and its organic moiety. It catalyses the conversion of lactose into glucose and galactose. The are commercially available and often referenced to as “lactases”. In a preferred embodiment, the beta-galactosidase used in the method of the invention is a neutral lactase. Currently, a number of neutral lactase products are sold by companies like DSM Food-Specialties, Godo, Chr. Hansen, Kerry, Novozymes, and others, under different brand names. All neutral lactases are produced by one of the two closely related yeast species Kluyveromyces lactis or Kluyveromyces marxianus (previously named Kluyveromyces fragilis). The enzymatic treatment step may be performed at above room temperature, up to a temperature of 65° C., and may be performed for a period of time sufficient to provide a high yield of glucose. The lactose or glucose concentration of the reaction may be monitored by a suitable probe. The galactose composition obtained by the enzymatic reaction typically contains D-galactose, and may also contain a mixture of galactose isomers.

As used herein, the term “glucose fermenting yeast” refers to a yeast that can grow on/in and metabolise glucose containing substrates and cannot metabolise (or only poorly metabolises) galactose. An example is yeast of the Saccharomyces spp., in particular a strain of Saccharomyces cerviscae. The yeast is generally added to the enzyme treated substrate in an amount sufficient to convert all or substantially all of the lactose in the substrate into glucose and galactose. The fermentation step may be performed at a temperature of 14° C. to 35° C. The fermentation step may be performed for 12 to 96 hours, depending on the concentration of glucose in the enzyme treated substrate. Obviously, if the step is performed at a lower temperature, the fermentation time may be longer, and vice versa.

### Exemplification

The invention will now be described with reference to specific Examples. These are merely exemplary and for illustrative purposes only: they are not intended to be limiting in any way to the scope of the monopoly claimed or to the invention described. These examples constitute the best mode currently contemplated for practicing the invention.

Referring to the drawings, one embodiment of the method of the invention is illustrated.

### Step 1

A lactose containing substrate is provided, containing 4.5% to 35% lactose by volume and having a pH of 3-9

### Step 2

A commercial beta-galactosidase enzyme, in this case a neutral Lactase is added to the substrate in an amount of 0.1% wt enzyme per L of permeate in a reaction vessel. The substrate and enzyme are then heated at 30-40° C. under gentle stirring until the concentration of glucose in the reaction vessel stabilises. This generally takes from 2 to 4 hours depending on the concentration of lactose in the substrate. At this stage, most of the lactose in the substrate has been converted into equal amounts of glucose and galactose.

### Step 3

Saccharomyces cerviscae cells are added to the reactor vessel, and the fermentation of the glucose is performed at 14-35° C. for 12-96 hours, under gentle stirring and under anaerobic conditions.

### Step 4 (Not Illustrated in the FIGURE)

The fermentation broth is removed from the reaction vessel and centrifuged to remove the sedimented yeast cells.

### Step 5

The supernatant in the centrifuge is removed and separated by reverse osmosis having a membrane with a pore size of <0.001 micron. This pore size allows permeation of small compounds such as ethanol and retains larger compounds such as galactose and minerals. The reverse osmosis step provides a retentate that is enriched in ethanol (e.g. 12-20% by volume) and also contains some trace compounds, and a retentate that contains all of the galactose. The reverse osmosis step takes place at pressures between 25 and 95 bar.

### Step 6

The retentate may be enriched in galactose by treatment to remove other compounds such as minerals. Methods of enriching the retentate may include treatment by nanofiltration or diafiltration, optionally including ion exchange chromatography or size exclusion chromatography, crystallisation or electrical dialysis.

The method of the invention represents a number of advantages compared to the methods of the prior art. As the method employs a lactose containing substrate, it provides a method of making ethanol and galactose that is not reliant on grain production, and is suitable for use in countries that have high levels of why production and low levels of grain. In addition, as the ethanol fraction is separated by a filtration technology (e.g. reverse osmosis), the method of the invention does not require expensive and energy demanding distillation processes. The method of the invention also provides an energy efficient method of producing galactose from readily available non-wheat substrates.

### Equivalents

The foregoing description details presently preferred embodiments of the present invention. Numerous modifications and variations in practice thereof are expected to occur to those skilled in the art upon consideration of these descriptions. Those modifications and variations are intended to be encompassed within the claims appended hereto.

1. A method of making an ethanol composition and a separate galactose composition by fermentation and comprising the steps of:
providing a lactose containing substrate this is not grain or derived from grain;
treating the lactose containing substrate with a beta-galactosidase enzyme to provide an enzyme treated substrate in which at least some of the lactose in the lactose containing substrate has been enzymatically converted into glucose and galactose;
fermenting in a fermentation medium comprising the enzyme-treated substrate a glucose fermenting yeast to provide a fermentation broth containing ethanol and galactose;
treating the fermentation broth to remove yeast cells; and
separating the treated fermentation broth into an ethanol composition and a separate galactose composition by reverse osmosis in which the permeate is the ethanol composition and the retentate is the galactose composition, wherein the method does not involve a distillation step.

2. The method according to claim 1, in which the step of separating the treated fermentation broth into an ethanol composition and a separate galactose composition by reverse osmosis comprises a single reverse osmosis separation stage.

3. The method according to claim 1, in which the ethanol composition produced has an ABV of 12% to 20%.

4. The method according to claim 1, in which the ethanol composition produced has an ABV of 5% to 25%.

5. The method according to claim 1, in which the treated fermentation broth has an ABV of 1% to 10%.

6. The method according to claim 1, in which the galactose composition is treated to enrich the galactose content of the galactose composition.

7. The method according to claim 1, in which the galactose composition is treated by one or more of nano-filtration, diafiltration, dialysis and size exclusion chromatography.

8. The method according to claim 1, in which the fermentation step comprises converting at least 80% by volume of the glucose in the enzyme-treated substrate into ethanol.

9. The method according to claim 1, in which the lactose containing substrate comprises at least 10% lactose (w/w).

10. The method according to claim 1, in which the lactose containing substrate comprises less than 1% glucose (w/w) and less than 1% galactose (w/w).

11. The method according to claim 1, in which the lactose containing substrate is dairy whey or a dairy whey derivative.

12. The method according to claim 1, in which the glucose fermenting yeast is a Saccharomyces spp.

13. The method according to claim 1, in which the glucose fermenting yeast is a strain of Saccharomyces cerviscae.

14. The method according to claim 1, in which the lactose containing substrate is demineralised.

15. The method according to claim 1, in which the lactose substrate is a whey derivative that has been treated to remove protein.

16. The method according to claim 1, that does not comprise a distillation or pervaporation step.

17. The method according to claim 1, in which the galactose composition is treated with a filtration step and a chromatography step.

18. The method according to claim 1, in which the lactose containing composition is dairy whey or a dairy whey derivative comprising at least 15% lactose by volume.

19. The method according to claim 1, in which the fermentation step is performed at 14-35° C. for 12-96 hours, under gentle stirring and under anaerobic conditions.

20. The method according to claim 1, in which the fermentation broth is centrifuged to provide a supernatant and sedimented yeast cells, the supernatant is separated by reverse osmosis at pressures between 25 and 95 bar having a membrane with a pore size of <0.001 micron to provide a permeate that is enriched in ethanol (12-20% by volume) and also contains some trace compounds, and a retentate that contains all of the galactose.
